(19) United States
(12) Patent Application Publication
(10) Pub. No.: US 2008/0102036 A1
(43) Pub. Date: May 1, 2008

(54) Biocompatible Fluorescent Silicon Nanoparticles

(76) Inventors: Kirtland G. Poss, Marblehead, MA (US); Karen N. Madden, Sudbury, MA (US); Kevin Groves, Somerville, MA (US); Milind Rajopadhye, Westford, MA (US)

Correspondence Address: Goodwin Proctor, Exchange Place, 53 State Street, Boston, MA, 02109, US

(21) Appl. No.: 10/559,558

(22) PCT Filed: Jun. 4, 2004
(86) PCT No.: PCT/US04/18023

§ 371 Date: Jan. 5, 2007

Related U.S. Application Data
(60) Provisional application No. 60/475,802, filed on Jun. 4, 2003.

(51) Int. Cl. A61K 49/00; G01N 33/53; A61P 43/00

(52) U.S. Cl. 424/9.6; 435/7.2

(57) ABSTRACT

The invention features biocompatible fluorescent nanoparticle and their use in in vivo imaging methods.

### RELATED APPLICATIONS

This application claims the benefit of U.S. Provisional Application No.60/475,802, filed Jun. 4, 2003. The entire teachings of which are incorporated herein by reference.

### BACKGROUND OF THE INVENTION

Optical imaging is an evolving clinical imaging modality that uses penetrating lights rays to create images. Preferably, light in the red and near-infrared (NIR) range (600-1200 nm) is used to maximize tissue penetration and minimize absorption from natural biological absorbers such as hemoglobin and water. (Wyatt, Phil. Trans. R. Soc. London B 352:701-706, 1997; Tromberg, et al., Phil. Trans. R. Soc. London B 352:661-667, 1997).

Besides being non-invasive, optical imaging methods offer a number of advantages over other imaging methods: they provide generally high sensitivity, do not require exposure of test subjects or lab personnel to ionizing radiation, can allow for simultaneous use of multiple, distinguishable probes (important in molecular imaging), and offer high temporal and spatial resolution (important in functional imaging and in vivo microscopy, respectively).

In optical imaging, filtered light or a laser with a defined bandwidth is used as a source of excitation light. The excitation light travels through body tissues. When it encounters a reporter molecule (i.e., contrast agent or imaging probe), the excitation light is absorbed. The reporter molecule then emits light that has detectably different properties from the excitation light. The resulting emitted light then can be used to construct an image.

Most optical imaging techniques have relied on the use of organic and inorganic fluorescent fluorochrome molecules as the reporter molecule. More recently, quantum dots or semi-conductor nanoparticles have been synthesized and used for certain in vitro biological applications (U.S. Pat. Nos. 6,468,808; 6,194,213; and 6,251,303; Dubertret et al., Science 298:1759-1762, 2002; and Wu et al., Nature Biotech. 21:41-46, 2003; Quantum Dot Corporation, Hayward, Calif.). Because of their limited solubility in aqueous media, their biological applications are greatly restricted. Furthermore, the use of the above described quantum dots or semi-conductor nanoparticles for in vivo applications remains highly questionable because of toxicity issues surrounding the introduction of toxic heavy metals into living systems. (Derfus et al., Nanoletters 4: 11-18, 2004). While these materials (containing Cd, Se, Te, In, etc.) may be useful as in vitro reagents, their potential heavy metal toxicity essentially precludes human applications.

There exists a need for agents and methods for use in in vivo and in vitro imaging. Such agents preferably are biocompatible, are non-immnunogenic, non-toxic, and can be derivatized or conjugated with affinity ligands, for example, biological or targeting moieties.

### SUMMARY OF THE INVENTION

The present invention features compositions of biocompatible fluorescent silicon nanoparticles, and methods of making such nanoparticles. It is an object of the invention to provide such particles for use in biological and biomedical applications. In particular, the present invention provides biocompatible fluorescent silicon nanoparticle imaging probes that can be used unmodified, or optionally coated with one or more various chemical moieties, biologically relevant coatings, conjugated to biomolecules and/or quenchable/activatable/light-shifting moieties, and such probes can be used for both in vitro and in vivo optical molecular imaging.

It is another object of the present invention to provide in vivo fluorescent silicon nanoparticle imaging probes comprised of biocompatible fluorescent silicon nanoparticles. It is another object of the present invention to provide fluorescent silicon nanoparticle imaging probes that are stable under aqueous conditions. It is another object of the present invention to provide in vivo fluorescent silicon nanoparticle imaging probes that are stable under physiological conditions. It is another object of the present invention to provide fluorescent silicon nanoparticle imaging probes that have long-circulating half-lives (e.g., plasma half life greater than several hours) in vivo. It is another object of the present invention to provide fluorescent silicon nanoparticle imaging probes formulated for administration to an animal or human subject for use in vivo imaging applications.

Accordingly, in one aspect, the invention features a fluorescent silicon nanoparticle.

In another aspect, the invention features a fluorescent silicon nanoparticle chemically linked with a biocompatible coating, forming a biocompatible fluorescent silicon nanoparticle.

In another aspect, the invention features a fluorescent silicon nanoparticle chemically linked to a biomolecule, forming a biocompatible fluorescent silicon nanoparticle.

In another aspect, the invention features a fluorescent silicon nanoparticle chemically linked to a biocompatible coating and a biomolecule, forming a biocompatible fluorescent silicon nanoparticle.

In another aspect, the invention features a biocompatible fluorescent silicon nanoparticle comprising a biocompatible coating of a silane, or other biologically equivalent coating that has been chemically linked to the nanoparticle.

In another aspect, the invention features a biocompatible fluorescent silicon nanoparticle consisting of or comprising a first biocompatible coating of a silane and a second biocompatible coating comprising a polymer.

In another aspect, the invention features a biocompatible fluorescent silicon nanoparticle consisting of or comprising a biocompatible coating of a silane chemically linked to one or more biomolecules.

In another aspect, the invention features a biocompatible fluorescent silicon nanoparticle consisting of or comprising a first biocompatible coating of a silane chemically linked to a second biocompatible coating comprising a polymer to which one or more biomolecules have been chemically linked.

In one embodiment, the biocompatible fluorescent silicon nanoparticle is a fluorescent silicon nanoparticle imaging probe that can be in an unactivated state having little or no fluorescence emission, and which can be activated, for example, by contact or interaction with a biological target whereby fluorescence emission can be detected.

In another embodiment, the fluorescent silicon nanoparticle imaging probe accumulates in, or binds to, diseased tissue at a different rate than in normal tissue. The diseased tissue can be, for example, cancerous, and the fluorescent silicon nanoparticle imaging probe accumulates in malignant tissue at a different rate than in normal or benign tissue. The diseased tissue can also be diseased due to an inflammatory disease and the fluorescent silicon nanoparticle imaging probe accumulates in diseased tissue at a different rate than in normal or benign tissue.

In another aspect, the invention features an in vivo or in vitro optical imaging method comprising administering to a sample or subject fluorescent silicon nanoparticle imaging probes of the present invention; allowing time for the fluorescent silicon nanoparticle imaging probes to contact the target; illuminating the target with light of a wavelength absorbable by the fluorescent silicon nanoparticle imaging probes; and detecting the optical signal emitted by the fluorescent silicon nanoparticle imaging probes.

These steps can also be repeated at predetermined intervals thereby allowing for the evaluation of emitted signal of the fluorescent silicon nanoparticle imaging probes in a subject or sample over time. The emitted signal may take the form of an image. The subject may be a vertebrate animal, for example, a mammal, including a human. The animal may also be non-vertebrate, (e.g., C. elegans, Drosophila, etc.). The sample can include, without limitation, cells, cell culture, tissue sections, organs, organ sections, cytospin samples, or the like.

The invention also features an in vivo method for selectively detecting and imaging two or more fluorescent silicon nanoparticle imaging probes simultaneously. The method comprises administering to a subject two or more fluorescent silicon nanoparticle imaging probes, either at the same time or sequentially, whose optical properties are distinguishable. The method therefore allows the recording of multiple events or targets.

The invention also features an in vivo method for selectively detecting or imaging one or more fluorescent silicon nanoparticle imaging probes, simultaneously with one or more targeted or activatable optical imaging probes, or in a dual imaging protocol with magnetic resonance imaging, computed tomography (CT), X-ray, ultrasound, or nuclear medicine imaging modalities and their respective imaging agents. The method comprises administering to a subject one or more imaging probes, either at the same time or sequentially, including at least one fluorescent silicon nanoparticle imaging probe, whose properties are distinguishable from that of the others. A preferred dual imaging protocol is optical and magnetic resonance imaging using fluorescent silicon nanoparticle imaging probes sequentially or nearly simultaneously with magnetic resonance imaging agents, (for example, iron oxide based agents or gadolinium based agents such as gadopentetate). The method therefore, allows the recording of multiple events or targets using more than one imaging modality or imaging agent.

In another aspect, the invention features an in vitro optical imaging method comprising contacting the sample with fluorescent silicon nanoparticle imaging probes; allowing time for the probes to become activated or bind to a target of interest in the sample; optionally, removing the unbound probes; illuminating the target with light of a wavelength absorbable by the fluorescent silicon nanoparticle imaging probes; and detecting the optical signal emitted by the fluorescent silicon nanoparticle imaging probes.

After administration, detection can occur, for example, by in vitro methods, e.g., flow cytometry or by in vivo imaging methods, e.g., tomographic, catheter, planar/reflectance systems or endoscopic systems. In one embodiment, the fluorescent silicon nanoparticles (or imaging probes derived thereof) can be used to label a sample ex vivo. The sample, e.g., cells, can be derived directly from a subject or from another source (e.g., from another subject, cell culture etc.). The fluorescent silicon nanoparticle imaging probe can be mixed with the cells to effectively label the cells and the resulting labeled cells injected into a subject. This method can be used for monitoring trafficking and localization of certain cell types, including T-cells and stem cells, and other cell types. In particular, this method may be used to monitor cell-based therapies.

Another aspect of the invention features fluorescent silicon nanoparticles formulated in a pharmaceutical composition suitable for administration to animals, including human subjects. The pharmaceutical composition can include the fluorescent silicon nanoparticles and one or more stabilizers in a physiologically relevant carrier.

Another aspect of the invention features biocompatible fluorescent silicon nanoparticles formulated in a pharmaceutical composition suitable for administration to animals, including human subjects. The pharmaceutical composition can include the nanoparticles and one or more stabilizers in a physiologically relevant carrier.

In one embodiment, the stabilizer is preferably a low molecular weight carbohydrate. In another embodiment the stabilizer is a linear polyalcohol, such as sorbitol, and glycerol. In a still further embodiment, the stabilizer is mannitol. Other low molecular weight carbohydrates, such as inositol, may also be used. Physiologically relevant carriers can include water, saline, and may further include agents such as buffers, and other agents such as preservatives that are compatible for use in pharmaceutical formulations.

The invention also features a method of gene sequence recognition using fluorescent silicon nanoparticles, labeled nucleic acid recognition molecules, including DNA, RNA, modified nucleic acid, PNA, molecular beacons, or other nucleic acid binding molecules (for example, small interfering RNA or siRNA). The method includes the use of one or more fluorescent silicon nanoparticles, together with techniques such as hybridization, ligation, cleavage, recombination, synthesis, sequencing, mutation detection, real-time polymerase chain reactions, in situ hybridization, and the use of microarrays. For example, for detecting a single stranded nucleic acid (e.g., mRNA, cDNA or denatured double-stranded DNA) in a sample, via nucleic acid hybridization principles, a fluorescent silicon nanoparticle chemically linked to a single-stranded nucleic acid is contacted with a sample containing one or more single stranded nucleic acids and the fluorescence of the fluorescent silicon nanoparticle is detected, wherein the presence or level of fluorescence signal emitted by the fluorescent silicon nanoparticle indicates the presence or amount of nucleic acid in the sample.

The optical signal generated by the fluorescent silicon nanoparticle imaging probes, or derivatives thereof, whether collected by tomographic, reflectance, planar, endoscopic, microscopic, surgical goggles, video imaging technologies, or other methods such as microscopy including intravital and two-photon microscopy, and whether used quantitatively or qualitatively, is also considered to be an aspect of the invention.

Another aspect of the invention features a kit, which includes the fluorescent silicon nanoparticle imaging probes, and optionally, instructions for using the nanoparticles for in vivo or in vitro imaging methods. The kit optionally can include components that aid in the use of the fluorescent silicon nanoparticle imaging probes for the disclosed methods, such as buffers, and other formulating agents; alternatively, the kit can include medical devices that aid in the administration of the fluorescent silicon nanoparticle imaging probes to subjects.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

FIG. 1 is an optical image of a mouse one minute after injection with a fluorescent silicon nanoparticle imaging probe of Example 6. The image was generated using Kodak 1D v.3.6.3 software (Kodak Imaging Systems). Four 15 second captures using appropriate excitation/emission filters were obtained to construct the fluorescent image.

FIG. 2 is an optical image of a mouse one minute after injection with the fluorescent silicon nanoparticle imaging probe of Example 21. The image was obtained as described in FIG. 1. The image was generated using Kodak 1D v.3.6.3 software (Kodak Imaging Systems). Four 15 second captures using appropriate excitation/emission filters were obtained to construct the fluorescent image

FIG. 3 is an optical image of a mouse one minute after injection with the fluorescent silicon nanoparticle imaging probe of Example 13. The image was generated using Kodak 1D v.3.6.3 software (Kodak Imaging Systems). Four 15 second captures using appropriate excitation/emission filters were obtained to construct the fluorescent image.

FIG. 4 is an optical image of a mouse one minute after injection with the fluorescent silicon nanoparticles imaging probes of Example 17. The image was generated using Kodak 1D v.3.6.3 software (Kodak Imaging Systems). Four 15 second captures using appropriate excitation/emission filters were obtained to construct the fluorescent image.

### DETAILED DESCRIPTION OF THE INVENTION

A description of preferred embodiments of the invention follows.

The present invention is based on fluorescent silicon nanoparticles that are suitable for in vitro and in vivo biological applications and methods for their uses. The fluorescent silicon nanoparticles in some embodiments are not chemically modified after synthesis. In other embodiments, the fluorescent silicon nanoparticles are further modified with one or more coating agents, e.g., a biocompatible coating, which may be optionally linked to a biomolecule. Alternatively, the biomolecule may be linked to the fluorescent silicon nanoparticle (without the biocompatible coating). The fluorescent silicon nanoparticles in any of these forms, may be fer formulated into fluorescent silicon nanoparticle imaging probes for use with in vitro and in vivo imaging applications.

The coatings, e.g., the biocompatible coating and the optional biomolecule, can be attached to the fluorescent silicon nanoparticle through one or more of a variety of chemical linkages. When the biocompatible fluorescent silicon nanoparticles comprise both a biocompatible coating and a biomolecule, the biomolecule can be linked to either the fluorescent silicon nanoparticle or the biocompatible coating, or to both the fluorescent silicon nanoparticle and the biocompatible coating.

The fluorescent silicon nanoparticle imaging probes have numerous advantages over other types of imaging probes. For example, the fluorescent silicon nanoparticles have a broad excitation spectrum, a narrow emission spectrum, are stable in biological milieu, show resistance to photobleaching, and preferably have NIR fluorescence capability.

A “fluorescent silicon nanoparticle” is a nanoparticle comprising silicon in a form that has fluorescent properties. Aggregates of crystalline silicon (as multiple or single crystals of silicon), porous silicon, or amorphous silicon, or a combination of these forms, can form the nanoparticle. Preferred fluorescent silicon nanoparticles have a diameter between about 0.5 nm to about 25 nm, more preferably between about 2 nm and about 10 nm. The size of fluorescent silicon nanoparticles can be determined by laser light scattering or by atomic force microscopy or other suitable techniques.

Fluorescent silicon nanoparticles can have excitation and emission spectra from about 200 to about 2,000 nm, however, preferred fluorescent silicon nanoparticles have excitation and emission maximum between about 400 nm and about 1,200 nm (and preferably between about 500 nm-900 nm, for example, about 500 nm-600 nm, about 600 nm-700 nm, about 700 nm-800 nm, or about 800 nm-900 nm). In a further embodiment, the fluorescent silicon nanoparticles also have extinction coefficients of at least 50,000 M⁻¹ cm⁻¹ in aqueous medium. It will be appreciated by one of skill in the art that the use of fluorescent silicon nanoparticles with excitation and emission wavelengths in other spectrums can also be employed in the compositions and methods of the present invention. For example, in certain embodiments, the particles can have excitation approximately about 300-350 nm, and emission approximately about 400-450 nm.

Preferred fluorescent silicon nanoparticles also have the following properties: (1) high quantum yield (e.g., quantum yield greater than 5% in aqueous medium), (2) narrow emnission spectrum (e.g., less than 75 nm; more preferably less than 50 nm), (3) spectrally separated absorption and emission spectra (e.g., separated by more than 20 nm; more preferably by more than 50 nm), (3) have high chemical stability and photostability (e.g., retain fluorescent properties after exposure to light), (4) are biocompatible (see below) or can be made more biocompatible; (5) are non toxic or minimally toxic to cells or subjects at doses used for imaging protocols, (as measured for example, by LD₅₀ or irritation studies, or other similar methods known in the art) and/or (6) have commercial viability and scalable production for large quantities (e.g., gram and kilogram quantities).

The fluorescent silicon nanoparticles may be obtained from any method that provides fluorescent silicon particles having the specifications as detailed above or provides fluorescent silicon nanoparticles that can be modified to specifications above. Methods known in the art include the synthesis and manufacture of fluorescent silicon nanoparticles as porous silicon, crystalline silicon, and/or amorphous silicon (fluorescent silicon nanoparticles that are neither crystalline nor porous).

Fluorescent silicon nanoparticles can be produced by electrochemical etching of silicon wafers (see, e.g., Li et al., (Langmuir 19: 8490-8496, 2003) which produces fluorescent silicon nanoparticles having micropores, which are generally called porous. Fluorescent silicon nanoparticles may also be produced by solution chemistry routes such as those described by Pettigrew (see Chem. Mater. 14:4005-4011, 2003), Kauzlarich et al. (see PCT Application WO 03/025260); or by Harwell (see PCT Application WO 01/14250) and result in fluorescent silicon nanoparticles having distinct crystal structures, generally called crystalline. Other methods of producing fluorescent silicon nanoparticles include the sonochemical approach by Dhas et al. (Chem Mater. 10:3278-3281, 1998), or gas phase decomposition of organic silicon compounds (see, e.g., Littau, K. A, et al., J. Phys. Chem. 87:1224,1993; FojtikA, et al., Chem. Phys. Lett. 221:363, 1994). Particles produced by these routes may have characteristics of both porous and crystalline silicon. Once obtained, these “native” fluorescent silicon nanoparticles (i.e. fluorescent silicon nanoparticles without further chemical modification) may be formulated as fluorescent silicon nanoparticle imaging probes for use in imaging protocols, or further synthesized with biocompatible coatings and/or biomolecules (collectively “coating agents”) that are chemically linked to the surface on the fluorescent silicon nanoparticles. These coating agents may provide active sites for linking chemistry, e.g, another biocompatible coating and/or biomolecule. Alternatively, the coating agent may include a biocompatible coating without further reactive sites. Examples of coating agents are provided below and in the Examples.

The native fluorescent silicon nanoparticles themselves can be “biocompatible” within the definition of biocompatible provided herein, i.e., water soluble or dispersible; or dispersible in a physiologically relevant media; non-immunognenic; and minimally toxic to living cells, tissues, organisms or animals. The terms “biocompatible coatings” and “biomolecules” refer to modifications of the fluorescent silicon nanoparticles with coating agents that are natural and/or synthetic chemical moieties. These coating agents are chosen to render the native fluorescent silicon nanoparticles more “biocompatible”, that is, e.g., more water soluble, or more dispersible in media for administration, or less immunogenic, or less toxic, or with altered biodistribution and phamarcokinetics when compared to the native fluorescent silicon nanoparticles. Similarly, the biocompatible coating agents can be chosen to reduce the nonspecific binding, and/or alter pharmacokinetics or biodistribution of the native fluorescent silicon nanoparticles. Additionally, biocompatible coating agents may be chosen to render the fluorescent silicon nanoparticle capable of functioning or existing in contact with biological fluids and/or tissue of a living organism; they may increase the specific binding of the fluorescent silicon nanoparticle to a target, and/or increase accumulation of the fluorescent silicon nanoparticle at a site. For example, ether groups in the linker chain of the coating agent may minimize plasma protein binding; a coating of methoxypolyethylene glycol (MPEG) or a peptide chain from about 1 to about 10 amino acid residues, may function to modify the pharmacodynamics and blood clearance rates of the fluorescent silicon nanoparticle imaging probes in vivo. Other biocompatible coating agents may be chosen to accelerate the clearance of the fluorescent silicon nanoparticle imaging probe from background tissue, such as muscle or liver, and/or from the blood, thereby reducing the background interference and improving image quality. Additionally, the coating agent may also be used to favor a particular route of excretion, e.g., via the kidneys rather than via the liver.

The biocompatible modifications may also aid in formulating fluorescent silicon nanoparticle imaging probes in pharmaceutical compositions or may be used to alter or preserve the optical properties of the compounds.

Thus, a “biocompatible fluorescent silicon nanoparticle” is a native fluorescent silicon nanoparticle to which one or more coating agents are chemically linked. Optionally, the native fluorescent silicon nanoparticle may be chemically linked directly to one or more biomolecules, or chemically linked to the fluorescent silicon nanoparticle through the biocompatible coating. The biocompatible coating and biomolecules are chosen and chemically linked to the fluorescent silicon nanoparticle so as to render the fluorescent silicon nanoparticle with altered properties over those of the native fluorescent silicon nanoparticles when used in the methods described herein.

The biocompatible fluorescent silicon nanoparticle has an estimated overall size from about 2 nm to about 100 nm, preferably from about 5 nm to about 100 nm. Preferably the biocompatible fluorescent silicon nanoparticles can be degraded in vivo into non-toxic components or be excreted, partially or in total.

A “biocompatible coating” is a coating agent that modifies or optimizes the fluorescent silicon nanoparticle as described above. There are several factors to consider when choosing a biological coating including, but not limited to, biocompatibility (see above), ease and reproducibility of fluorescent silicon nanoparticle surface modification, presence of reactive groups for chemically linking biomolecules or other biocompatible coatings, commercial availability, and cost.

Preferably, the biocompatible coating does not adversely affect the fluorescent properties of the fluorescent silicon nanoparticle (e.g., it does not quench the fluorescence, or shift the fluorescence outside the preferred excitation or emission spectra). Additionally, the biocompatible coating may preserve the fluorescent properties of the fluorescent silicon nanoparticles by insulating the nanoparticles from fluorescent diminishing moieties, such as water. In Examples 13 and 19, native fluorescent silicon nanoparticles coated with 4-(mPEGthio)butane retain their fluorescence for at least seven days in aqueous media. In certain embodiments, the biocompatible coating may shift the optical properties of the fluorescent silicon nanoparticles, for example, where the native fluorescent nanoparticles have excitation/emission spectra outside a preferred range, the biocompatible coating may be selected to adjust the spectra to the preferred ranges, (e.g., see Example 3).

Preferably, the biocompatible fluorescent silicon nanoparticle is water soluble or water dispersible (i.e., sufficiently soluble or suspendable in aqueous or physiologically relevant media). The biocompatible coating may be chemically linked to multiple sites (e.g, surface groups) on the native fluorescent silicon. nanoparticle. Importantly, more than one biocompatible coating may be chemically linked to the native fluorescent silicon nanoparticle to form more than one coat or layer or cage on the nanoparticle.

The biocompatible coating may be a polymer, including natural polymers, or synthetic polymers, or derivatives of each. The polymer may be grafted, linear, branched or arborized/dendrimerized. Examples of natural polymers include polysaccharides, such as dextran, proteins, such as albumin, peptides and polyamino acids, such as polylysine. A synthetic polymer is obtained from nonbiological syntheses, by using standard polymer chemistry techniques known to those in the art to react monomers into polymers. The polymers may be homopolymers, (i.e., synthesized from a single type of monomer), or co-polymers, (i.e., synthesized from two or more types of monomers). The polymers can be crosslinked (e.g., a polymer in which functional groups on a polymer chain and/or branches have reacted with functional groups on another polymer to form polymer networks) or non-cross-linked (e.g., few or no individual polymer chains have reacted with the fimctional groups of another polymer chain to form the interconnected polymer networks). Synthetic, biocompatible polymers are discussed generally in Holland et al., “Biodegradable Polymers,” Advances in Pharmaceutical Sciences 6:101-164, 1992, and U.S. Pat. No. 5,593,658. Preferred polymers have a molecular weight of about 5,000-10,000 daltons. The polymers may be attached directly to the native nanoparticle, or attached to coating agents through reactive groups on the coating agents. Alternatively, the polymers may be formed in situ, i.e., added as monomers to the fluorescent silicon nanoparticle solution, e.g. as an acrylate, and polymerized e.g., with standard polymerization chemistries, to form the polymer in the presence of the fluorescent silicon nanoparticles.

Useful types of polymers include polypeptides, polyamino acids, diaminocarboxylate, copolymers, polyethyleneamines, polysaccharides, aminated polysaccharides, aminated oligosaccharides, polyamidoamines, polyacrylic acids, polyalcohols, polyoxyethytene sorbiian esters, polyoxyethytene and polyoxypropylene derivatives, polyoxyl stearates, polycaprolactones, polyanhydrides, polyalkylcyanoacrylates, polyglycerol surfactants, polycaprolactones, polyanhydrides, polymethylmethacrylate polymers, starch derivatives, dextran and derivatives thereof (i.e., carboxydextran, carboxymethyldextran, reduced carboxymethyldextran), fatty acids, their salts and derivatives, mono-, di-, and triglycerides and their derivatives, and poly-carboxylic acids. Preferred polymers include polyethylene oxide, poly(vinyl pyrrolidone), poly (methacrylic acid), poly(acrylic acid), poly(hydroxyethylmethacrylate, poly(vinyl alcohol) and natural polymers such as dextran.

Other useful types of biocompatible coatings include silanes, which are commercially available. Preferred silanes are organosilanes that contain a reactive functional group. For bifinctional silanes, preferred additional reactive functional groups are amino, phosphate, mercapto, isocyanoto or aldehyde groups that can be used to react with appropriate functional groups on coating agents. Useful types of silanes include alkoxy silanes (including methoxy and ethoxy), halogenated silanes, including bromosilanes and chlorosilanes. Alkoxy silanes and aldehydic alkoxy silanes are preferred.

Other preferred silanes are aminoalkyl-trialkoxysilanes (such as 3-amino-tripropyltrimethoxy silane (APTMS); 3-aminopropyltrimethoxysilane; or 2-aminoethyltrimethoxysilane), trimethylsilyformic acid, 3-(trichlorosilyl) butanoic acid, 1,1,1-trichloro-N-(trimethylsilyl) silanamine, trichlorovinlysilane (TCVS), vinyltrimethoxysilane, (3-glycidoxypropymethyldiethoxysilane, 3-glycidoxypropyltrimethoxysilane, 3-isocyanatopropyltriethoxysilane, and diethylphosphatoethyltriethoxysilane. The silane coating may be deposited as a monolayer or in multilayers. Silanes can also be crosslinked to cage the fluorescent silicon nanoparticle.

Other useful types of biocompatible coatings include polyethylene glycol (PEG), methoxypolyethylene glycol (MPEG), methoxypolypropylene glycol, polyethylene glycol-diacid, polyethylene glycol monoamine, MPEG monoamine, MPEG hydrazide, MPEG imidazole. Alkenes, and alkynes, such as hexene, may be used.

A “biomolecule” is a moiety that can be chemically linked to the fluorescent silicon nanoparticles of the present invention and changes or enhances accumulation, biodistribution, elimination, targeting, binding, and/or recognition of the fluorescent silicon nanoparticle nanoparticle or other properties as described above. Biomolecules include but are not limited to antibodies and fragments thereof, proteins, peptides, antibodies (or antigen-binding antibody fragments, such as single chain antibodies), glycoproteins, ligands for cell receptors, polysaccharides, cell receptors themselves, enzyme substrates, enzyme cofactors, biotin, hormones, neurohormones, neurotransmitters, growth factors, cytokines, lymphokines, lectins, selectins, toxins, and carbohydrates. Other targeting and delivery approaches using various biomolecules can also be used, such as folate-mediated targeting (Leamon & Low, Drug Discovery Today, 6:44-51, 2001), transferrin, vitamins, carbohydrates and ligands that target internalizing receptors, including, but not limited to, asialoglycoprotein receptor, somatostatin, nerve growth factor, oxytocin, bombesin, calcitonin, arginine vasopressin, angiotensin II, atrial natriuretic peptide, insulin, glucagons, prolactin, gonadotropin, various opioids and urokinase-type plasminogen activator. Also included are membrane, transmembrane, and nuclear translocation signal sequences, which can be derived from a number of sources including, without limitation, viruses and bacteria.

The biomolecules can be directly chemically linked to the surface of the native fluorescent silicon nanoparticle directly, or to a biocompatible coating on a fluorescent silicon nanoparticle. Preferably, chemically linking one or more biomolecules to the particle does not alter the activity of the biomolecules. One or more biomolecules, including different biomolecules, can be chemically linked to the fluorescent silicon nanoparticles. Some preferred embodiments have more than one biomolecule attached to a fluorescent silicon nanoparticle, where the biomolecules are all the same or different.

“Chemically linked” means connected by an attractive force between atoms strong enough to allow the combined aggregate to function as a unit. This includes, but is not limited to, chemical bonds such as covalent bonds, non-covalent bonds such as ionic bonds, metallic bonds, and bridge bonds, hydrophobic interactions, hydrogen bonds, and van der Waals interactions. This also includes crosslinking or caging.

A “fluorescent silicon nanoparticle imaging probe” is any fluorescent silicon nanoparticle that can be used for biological imaging applications, including in vitro and in vivo imaging applications. This includes, but is not limited to, native fluorescent silicon nanoparticles and biocompatible fluorescent silicon nanoparticles.

A “biological target” includes a biological moiety, including, but not limited to cells, proteins, nucleic acids, genes, proteins, enzymes and tissues. A biological target further includes organs, organ systems, organ sections, vessels; cell, tissue and organ receptors; and cellular or metabolic pathways.

### Synthesis of Fluorescent Silicon Nanoparticles

Methods for the synthesis and manufacture of porous silicon nanoparticles having fluorescent and fluorescence properties are known in the art (e.g., U.S. Pat. Nos. 5,427,648, 5,852,346 and 5,272,355). For example, porous silicon can be produced by electrochemically etching the surface of a crystalline silicon wafer. This is typically achieved by using solutions containing hydrofluoric acid and by applying an electrochemical current. Fluorescent silicon nanoparticles are typically produced from the etched silicon wafer surface by ultrasonic fracture, mechanical grinding or by lithographic methods. By varying different etching parameters, such as the duration of etching, electrochemical current, characteristics of the silicon wafer and composition of the etching solution, the size and porosity of the particles can be controlled, and hence the fluorescent properties of the particles (see Li et al., Langmuir 19: 8490-8496, 2003).

Other methods of producing silicon fluorescent nanoparticles include high temperature decomposition of disilane (Littau et al. J. Phys. Chem. 97:1224-1230, 1993); laser vaporization controlled condensation of silane (Carlisle et al. Chem. Phys. Lett. 326:335-340, 2000); and the conversion of diphenylsilane into silicon nanocrystals at high temperature (500° C.) and pressure (345 bar) in supercritical organic solvents (Ding et al., Science 296: 1293-1297, 2002).

Crystalline silicon fluorescent nanoparticles have been produced by reacting silicon Zintl salts with silicon halides, solution reduction of silicon Zintl salts with silicon halides, solution reduction of silicon halides by sodium, lithium naphthalenide or hydride reagents, reduction of Si(OEt)₄ with sodium; and reacting silicon halide with a reducing agent in organic solvent at ambient conditions. These nanoparticles can be frther surface modified. (Pettigrew, Chem. Mater. 14: 4005-4011, 2003; Kauzlarich et al., PCT Application No. WO 03/025260; Harwell, PCT Application No. WO 01/14250).

Other methods, such as the sonochemical approach by Dhas et al. (Chem. Mater. 10:3278-3281, 1998) use a combination of solution chemistry methods and mechanical means to obtain silicon fluorescent nanoparticles with desired properties. Particles of various sizes can be purified using techniques known in the art, such as size exclusion chromatography, density gradient centrifugation and colloidal separations techniques. Preferred fluorescent silicon nanoparticles preparations are monodisperse, i.e., have similar a similar size or composition.

The fluorescent silicon nanoparticle surface can be comprised of elemental silicon, silicon dioxide, silicon oxide, silicon halide, silicon hydroxyl, silicon hydride, other silicon compounds, or any combination thereof. The composition of the surface of the particle can be controlled by using techniques known in the art. For example, native fluorescent silicon nanoparticles may react with air or water under ambient conditions to form a thin surface of silicon dioxide, which may hydrate and render particles hypdrophilic. Methods to prevent oxidation and stabilize silicon surfaces are known in the art (e.g., Stewart et al., Phys. Stat. Sol. 182:109-115, 2000).

The native fluorescent silicon nanoparticles may stored for later use, preferably, dry and under an inert atmosphere (e.g., nitrogen); optionally, the native fluorescent silicon nanoparticles may be stored in solutions of chloroform, toluene, or alcohols, or in a suspension of mineral oil, or glycerin.

To improve their dispersion and or dissolution properties in aqueous or physiologically relevant media, the native fluorescent silicon nanoparticles may be stored or formulated in solutions containing low molecular weight carbohydrates, such as mannitol. These solutions may stabilize the fluorescence properties and permit the use of the native fluorescent silicon nanoparticles as biocompatible in imaging protocols without further surface modifications. Although mannitol is most preferred, other low molecular weight carbohydrates may be used. The low molecular weight carbohydrates have a molecular weight less than about 5,000 daltons, preferably about 1,000 daltons or less. Examples include low molecular weight dextrans or inositol, with the more preferable agents being linear polyalcohols, such as sorbitol, and glycerol. The preferred concentration is about 10% (w/v) in the media. Additionally, use of these low molecular weight carbohydrates in colloidal solutions has been shown to stabilize the suspensions against unwanted physical changes that may result from environmental conditions, e.g., prolonged or inappropriate storage, or that result from processing the materials for use in animals and humans, e.g., sterilization procedures. (See U.S. Pat. No. 5,248,492).

For example, Example 19 uses 10% (w/v) mannitol in PBS (phosphate buffered saline) to disperse the mPEG-thiobutane coated fluorescent silicon nanoparticles after synthesis to stabilize the fluorescence of the fluorescent silicon nanoparticles. In Example 21, native fluorescent silicon nanoparticles are dispersed in solution of mannitol, 3% dimethylsulfoxide (DMSO), and phosphate buffered saline (PBS), and the resulting fluorescent silicon nanoparticle imaging probe is administered to mice prior to imaging. Examples 20 and 23 also use mannitol as a dispersing agent for the coated fluorescent silicon nanoparticle imaging probes.

### Biocompatible Fluorescent Silicon Nanoparticles and Biomolecule Conjugates

In the practice of the present invention, the biomolecules and biocompatible coatings can be chemically linked to the fluorescent silicon nanoparticles by methods known in the art for chemically linking two or more moieties. Techniques and methods are known in the art for how to chemically link biocompatible coatings and biomolecules to different types of nanoparticles and surfaces and these basic techniques can be applied to fluorescent silicon nanoparticles (see, for example, U.S. Pat. Nos. 5,782,908, 4,118,485, 4,673,584, and Bioconjugate Techniques, Academic Press, New York, 1996).

For example, to chemically link a silane to a fluorescent silicon nanoparticle, a silane is preferably dissolved in a suitable solvent to form a solution, which is then placed in contact with the fluorescent silicon nanoparticle surface. Suitable solvents may include, for example, chloroform, methylene chloride and aqueous solutions of alcohols. The concentration of silane in solution is preferably approximately 0.1% to 10% (v/v). Generally, the silane solution remains in contact with the particle surface for about 0.5 to 6 hours at ambient temperatures. Depending on the nature of the silane and functional groups on the silane, other biocompatible coatings and/or biomolecules can then be chemically linked to the silane coated fluorescent silicon nanoparticle. Optionally, the biomolecule can be first linked to the silane; the complex is then reacted with the fluorescent silicon nanoparticle. This technique is useful where the linking chemistry employs solvents detrimental to the fluorescent properties of the fluorescent silicon nanoparticle.

As an illustrative example, biomolecules can be directly reacted to an aldehydic silane coated fluorescent silicon nanoparticle under aqueous conditions. The aldehyde groups on the silane coated fluorescent silicon particle react with primary amine groups on biomolecules resulting in covalent attachment of the biomolecule to the fluorescent silicon nanoparticle.

Linkers or spacers may be used to chemically link biomolecules or biocompatible coatings to the fluorescent silicon nanoparticles of the present invention. Usefuil linker moieties include both natural and non-natural amino acids and nucleic acids, as well as synthetic linker molecules. These linkers can be homofunctional linkers or heterofunctional linkers. There is no particular size or content limitations of the linker or spacer. Particularly useful linker moieties are bifunctional crosslinkers such as N-succinimidyl 3-(2-pyridyldithio)propionate (SPDP), long chain-SPDP, maleimidobenzoic acid-N-hydroxysuccinimide ester (S), succinimidyl trans-4-(maleimidylmethyl)cyclohexane-1-carboxylate (SMCC), and others that are known in the art and are commercially available from vendors such as Pierce Chemical Company, Rockford, Ill.

The biocompatible coating or biomolecule can be functionalized for attachment to the fluorescent silicon nanoparticle. For example, 3-aminopropyl trimethoxy silane (APS) is a silane that can be used to coat fluorescent silicon nanoparticles. To functionalize the silane coated nanoparticle so that it can form a covalent bond between other biocompatible coatings and/or biomolecules (e.g., the primary amine of a peptide), a heterobifunctional crosslinker, such as N-5-azido-2-nitrobenzoyloxysuccinimide (ANB-NOS) can be used.

In preferred embodiments, a fluorescent silicon nanoparticle is reacted with bromopropylsilane, cyanopropylsilane or thiopropylsilane, and then heated to form a silane coated fluorescent silicon nanoparticle with the silane molecules on the surface of the fluorescent silicon nanoparticle crosslinked, i.e., a silane “caged” particle. The functional groups e.g., bromo, cyano or thiol groups, on the silane molecules can then be used to attach additional biocompatible coatings and/or biomolecules. For instance, a bromopropylsilane caged particle can be directly reacted with methoxy polyethylene glycol (mPEG) succinimidyl succinate to form an mPEG coated fluorescent silicon nanoparticle (see Example 18); or a mercaptopropyl silane capped caged particle can be reacted with a biomolecule such as human EGF to form an EGF-coated fluorescent silicon nanoparticle (see Examples 9 and 16).

In another preferred embodiment, a fluorescent silicon nanoparticle can be reacted with thionylchloride to functionalize the surface of the fluorescent silicon nanoparticle to which another biocompatible coating, such as dextran, carboxydextran, carboxymethyldextran, reduced carboxymethyl dextran (see U.S. Pat. No. 6,599,498) or hydroxyl-polyethylene glycol can then be attached.

Another approach is to crosslink a dextran coated fluorescent silicon nanoparticle with epichlorohydrin and to introduce amine groups on the surface by reacting the dextran with ammonia (see Josephson et al., Bioconjug Chem 10:186-91, 1999; Josephson et al., Angwandte Chemie 40:3204-3206, 2001). The amine groups can be used to react with many bifunctional cross linker reagents that consist of N-hydroxysuccinimide esters that react first with an amine group and have a second group that reacts with a sulfhydryl group on a biomolecule, such as a cysteine molecule.

Unreacted biocompatible coatings and/or biomolecules can be separated from the desired fluorescent silicon nanoparticle product, and this can be accomplished by gel filtration, ultrafiltration, dialysis, or other chromatography methods.

### Fluorescent Silicon Nanoparticle Inaging Probes

The fluorescent silicon nanoparticles can be used as optical reporters on or in a number of different fluorescent silicon nanoparticle imaging probes, including (1) probes that become activated after target contact (e.g., binding or interaction) (Weissleder et al., Nature Biotech., 17:375-378, 1999; Bremer et al., Nature Med., 7:743-748, 2001), (2) wavelength shifting probes (Tyagi et al., Nat. Biotechnol., 18:1191-1196, 2000), (3) multicolor fluorescence probes (Tyagi et al., Nat. Biotechnol., 16:49-53, 1998), or (4) probes that have high binding afinity to targets, i.e., that remain within a target region while non-specific probes are cleared from the body (Achilefu et al., Invest. Radiol., 35:479-485, 2000; Becker et al., Nature Biotech. 19:327-331, 2001; Bujai et al., J. Biomed. Opt. 6:122-133,2001; Ballou et al. Biotechnol. Prog. 13:649-658, 1997; and Neri et al., Nature Biotech. 15:1271-1275, 1997), or (5) as an imaging probe by itself that preferentially accumulates in diseased tissue at a different rate compared to normal tissue (Reynolds et al., Photochem. Photobiol. 70:87-94, 1999; Becker et al., Photochem. Photobiol. 72:234-241, 2000).

By “activation” of a fluorescent silicon nanoparticle imaging probe after target contact or interaction is meant a change to the probe that alters a detectable property, e.g., an optical property, of the probe. This includes, but is not limited to, a modification, alteration, or binding (covalent or non-covalent) of the probe that results in a detectable difference in properties, e.g., optical properties of the probe, e.g., changes in the fluorescence signal amplitude (e.g., dequenching and quenching), change in wavelength, fluorescence lifetime, spectral properties, or polarity. Optical properties include wavelengths, for example, in the visible, ultraviolet, NE, and infrared regions of the electromagnetic spectrum. Activation can be, without limitation, by enzymatic cleavage, enzymatic conversion, phosphorylation or dephosphorylation, conformation change due to binding, enzyme-mediated splicing, enzyme-mediated transfer, hybridization of complementary DNA or RNA, analyte binding, such as association with an analyte such as Na⁺, K⁺, Ca²⁺, Cl⁻, or another analyte, change in hydophobicity of the environment and chemical modification.

In another embodiment, a quencher molecule is used to quench the fluorescent signal of the fluorescent silicon nanoparticle imaging probe. The quencher molecule is situated such that it quenches the optical properties of the fluorescent silicon nanoparticle imaging probe. The quencher can be attached, for example, to a portion of the fluorescent silicon nanoparticle (e.g., to the nanoparticle, the biocompatible coating, or to the biomolecule). Upon activation of the fluorescent silicon nanoparticle imaging probe, the fluorescent silicon nanoparticle imaging probe is de-quenched. By adopting these activated and unactivated states of a fluorescent silicon imaging probe in a living animal or human, the probe will exhibit different signal intensities, depending on whether the probe is active or inactive. It is therefore possible to determine whether the probe is active or inactive in a living organism by identifying a change in the signal intensity of the fluorescent silicon nanoparticle imaging probe, the quencher molecule, or a combination thereof. In addition, because the fluorescent silicon nanoparticle imaging probe can be designed such that the quencher molecule quenches the fluorescent silicon nanoparticle imaging probe when the probe is not activated, the fluorescent silicon nanoparticle imaging probe can be designed such that the fluorescent silicon nanoparticle imaging probe exhibits little or no signal until the probe is activated.

There are a number of quenchers available and known to those skilled in the art including, but not limited to 4-{[4-(Dimethylamino)-phenyl]-azo}-benzoic acid (DABCYL), QSY®-7 (9-[2-[(4-carboxy-1-piperidinyl)sulfonyl]phenyl]-3,6-bis(methylphenylamino)-xanthylium chloride) (Molecular Probes, Inc., OR), QSY®-33 (Molecular Probes, Inc., OR), and fluorescence dyes such as CyS and Cy5.5 (e.g., 2-[5-[3-[6-[(2,5-dioxo-1-pyrrolidinyl)oxy]-6-oxohexyl]-1,3-dihydro-1,1-dimethyl-6,8-disulfo-2H-benz[e]indol-2-ylidene]-1,3-pentadienyl]-3-ethyl-1,1-dimethyl-6,8-disulfo-1H-benz[e]indolium, inner salt) (Schobel, Bioconjugate 10:1107, 1999). Methods for attaching a quencher to a molecule, for example, a probe are known in the art.

Other quenching strategies can be used, for example, using various solvents to quench fluorescence of the fluorescent silicon nanoparticle imaging probe.

The fluorescent silicon nanoparticle imaging probes may be also be used for gene sequence recognition, labeled nucleic acid recognition molecules, including DNA, RNA, modified nucleic acid, PNA, molecular beacons, or other nucleic acid binding molecules (for example, small interfering RNA or siRNA), using techniques such as hybridization, ligation, cleavage, recombination, synthesis, sequencing, mutation detection, real-time polymerase chain reactions, in situ hybridization, and the use of microarrays. For example, for detecting a single stranded nucleic acid (i.e., mRNA, cDNA or denatured double-stranded DNA) in a sample, via nucleic acid hybridization principles, a fluorescent silicon nanoparticle chemically linked to a single-stranded nucleic acid is contacted with a sample containing one or more single stranded nucleic acids and the fluorescence of the fluorescent silicon nanoparticle imaging probe is detected, wherein the presence or level of fluorescence signal emitted by the fluorescent silicon nanoparticle imaging probe indicates the presence or amount of nucleic acid in the sample.

### Biological Properties

In preferred embodiments of the present invention, the in vivo half-life of the fluorescent silicon nanoparticle imaging probe is at least about 10 minutes, but more preferably 30 minutes to several hours. In other preferred embodiments of the invention, the in vivo half-life of the fluorescent silicon nanoparticle imaging probe is a time (for example, at least about one hour) sufficient to perform luminal delineating studies, such as gastrointestinal imaging or major vessel angiography, fluorescence (micro) angiography, perfusion and angiogenesis studies.

In preferred embodiments of the present invention, the fluorescent silicon nanoparticle imaging probe is water soluble or dispersible in aqueous media, and is non-toxic (e.g., has an LD₅₀ of greater than about 50 mg/kg body weight or higher). In other preferred embodiments of the present invention, the fluorescent silicon nanoparticle imaging probes do no have any phototoxic properties.

In some preferred embodiments of the present invention, the fluorescent silicon nanoparticle imaging probes show little serum protein binding affinity.

### Formulations

For in vivo use, the compositions may be provided in a formulation that is suitable for administration to animal, including human, subjects. The formulations include the fluorescent silicon nanoparticle imaging probes together with a physiologically relevant carrier suitable for the desired form and/or dose of administration. By “physiologically relevant carrier” is meant a carrier in which the fluorescent silicon nanoparticle imaging probe is dispersed, dissolved, suspended, admixed and is physiologically tolerable, i.e., can be administered to, in, or on the subject's body without undue discomfort, or irritation, or toxicity. The preferred carrier is a fluid, preferably a liquid, more preferably an aqueous solution; however, carriers for solid formulations, topical formulations, inhaled formulations, ophthalmic formulations, and transdermal formulations are also contemplated as within the scope of the invention.

Methods of administration include the oral, parenteral (e.g., intravenously, intramuscularly, subcutaneous, by injection, infusion, or implant), rectal, cutaneous, nasal, vaginal, inhalant, skin (patch), or percutaneously, ocular administration route. Thus, the composition may be in the form of, e.g., solid tablets, capsules, pills, powders including lyophilized powders, colloidal suspensions, microspheres, liposomes granulates, suspensions, emulsions, solutions, gels, including hydrogels, pastes, ointments, creams, plasters, irrigation solutions, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Germaro, Lippincott Williams & Wilkins, Philadelphia, and Encyclopedia of Pharmaceutical Technology, eds. J. Swarbrick and J. C. Boylan, 1988-1999, Marcel Dekker, New York, hereafter “Remington's”).

Pharmaceutically acceptable formulations can include carriers, adjuvants and vehicles that may contain one or more stabilizers, buffers, pH modifiers, tonicity adjusting agents (e.g. salts of plasma cations with appropriate counterions), preservatives, antimicrobial agents, and other formulating agents as known in the art and as needed for the specific formulation (see Remington's, supra). These agents aid in manufacturing and using the final product such as in the formulating of the product, including sterilization if necessary, stability and storage characteristics of the product, administration of the product, and lack of discomfort or toxicity to subject.

Carriers, adjuvants, and/or vehicles include, but are not limited to ion exchangers, alumina, aluminum stearate, lecithin, serum proteins such as albumin, buffer substances such as phosphate, glycine, sorbic acid, potassium sorbate, TRIS (tris(hydroxymethyl)amino methane), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block polymers, sugars such as glucose, and suitable cryoprotectants.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. Suitable antimicrobial preservative(s) include the parabens, (methyl, ethyl, propyl or butyl paraben or mixtures thereof); benzyl alcohol; phenol; cresol; cetrimide and thiomersal. Preferred antimicrobial preservative(s) are the parabens.

The term “pH-adjusting agent” means a compound or mixture of compounds useful to ensure that the pH of liquid or reconstituted powder formulations are within physiological acceptable limits (approximately from about pH 4.0 to about 10.5) for animal including human, administration. Suitable such pH-adjusting agents include pharmaceutically acceptable buffers, such as tricine, phosphate or TRIS [i.e., tris(hydroxymethyl) aminomethane], and pharmaceutically acceptable bases such as sodium carbonate, sodium bicarbonate or mixtures thereof.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose. Other pharmaceutically acceptable agents such as colorants, flavoring agents, plasticizers, humectants, and the like, may also be included in the formulation.

The formulation of the fluorescent silicon nanoparticle imaging probe can also include an antioxidant or some other chemical compound that prevents or reduces the degradation of the baseline fluorescence, or preserves the fluorescence properties, including, but not limited to quantum yield, fluorescence lifetime, and excitation and emission wavelengths. These antioxidants or other chemical compounds can include, but are not limited to melatonin, dithiothreitol (DTT), deferoxamine (DFX), methionine, DMSO, and N-acetyl cysteine.

The fluorescent silicon nanoparticle imaging probe and pharmaceutical compositions of the present invention can be administered orally, parentally, by inhalation, topically, rectally, nasally, buccally, vaginally, or via an implanted reservoir. The term “parental administration” includes intravenous, intramuscular, subcutaneous, intraarterial, intraarticular, intrasynovial, intrasternal, intrathecal, intraperitoneal, intracistemal, intrahepatic, intralesional, intracranial and intralymphatic injection or infusion techniques. The fluorescent silicon nanoparticle imaging probes can also be administered via catheters or through a needle to a tissue.

For injection, a sterile injectable preparation can be prepared by one skilled in the art according to techniques known in the art. Vehicles or solvents that can be used to make injectable preparations include sterile, pyrogen-free water for injection, Ringer's solution, isotonic sodium chloride solution, and D5W; saline (preferably balanced so that the final product for injection is isotonic); an aqueous solution of one or more tonicity-adjusting substances (e.g. salts of plasma cations with biocompatible counterions), sugars (e.g glucose or sucrose), sugar alcohols (e.g. sorbitol or mannitol), glycols (e.g. glycerol), or other non-ionic polyol. materials (e.g. polyethyleneglycols, propylene glycols and the like) In addition, oils such as mono- or di-glycerides and fatty acids such as oleic acid and its derivatives can be used.

For ophthalmic use, the pharmaceutical composition of the invention can be formulated as micronized suspensions in isotonic, pH adjusted sterile saline. Alternatively, the compositions can be formulated in ointments such as petrolatum.

For topical application, the new pharmaceutical compositions can also be formulated in a suitable ointment, such as petrolatum. Transdermal patches can also be used. Topical application for the lower intestinal tract or vagina can be achieved by a suppository formulation or enema formulation.

The pharmaceutical compositions described herein may be sterilized by the methods known in the pharmaceutical industry. The generally preferred methods include, autoclaving (subjecting the material to heat, generally 80° C. or higher for extended periods), aseptic preparations, and lyophilization (filter sterilization). For example, U.S. Pat. No. 6,599,498 describes methods of autoclaving a colloidal imaging agent using reduced carboxylated polysaccharides as excipients to prevent heat stress induced physical changes in the material. U.S. Pat. Nos. 4,827,945 and 5,055,288 use citrate as autoclaving excipients for a metal oxide imaging agent. Similarly, U.S. Pat. No. 5,102,652 adds low molecular weight carbohydrates to the formulation. Alternatively, U.S. Pat. No. 5,160,726, uses filter sterilization rather than heat to sterilize an injectable colloidal imaging agent.

For in vitro applications, the composition may be supplied as a powder for reconstitution, a liquid, including concentrated, or ready to use in appropriate buffer solutions, e.g., PBS.

### In Vitro Testing

After a fluorescent silicon nanoparticle imaging probe is designed, synthesized, and optionally formulated, it can be tested in vitro by one skilled in the art to assess its biological and performance characteristics. For instance, different types of cells grown in culture can be used to assess the biological and performance characteristics of the fluorescent silicon nanoparticle imaging probe. Cellular uptake, binding or cellular localization of the fluorescent silicon nanoparticle imaging probe can be assessed using techniques known in the art such as fluorescent microscopy. For example, fluorescent silicon nanoparticle imaging probes of the present invention can be contacted with a sample for a period of time and then washed to remove any free fluorescent silicon nanoparticle imaging probe. The sample can then be viewed using a fluorescent microscope equipped with appropriate filters matched to the optical properties of the fluorescent silicon nanoparticle imaging probe. Fluorescent microscopy of cells in culture is also a convenient means for determining whether uptake and binding occurs in one or more subcellular compartments. Tissues, tissue sections and other types of samples such as cytospin samples can also be used in a similar manner to assess the biological and performance characteristics of the fluorescent silicon nanoparticle imaging probe. Other fluorescent detection methods including, but not limited to flow cytometry, immunoassays, hybridation assays, and microarray analysis can also be used.

### Optical Imaging Methods

Although the invention involves novel fluorescent silicon nanoparticle imaging probes, general principles of fluorescence, optical image acquisition, and image processing can be applied in the practice of the invention. For a review of optical imaging techniques, see, e.g., Alfano et al., Ann. NY Acad. Sci. 820:248-270, 1997.

An imaging system useful in the practice of this invention typically includes three basic components: (1) an appropriate light source for fluorescent silicon nanoparticle imaging probe excitation, (2) a means for separating or distinguishing emissions from light used for fluorochrome excitation, and (3) a detection system. This system can be hand-held or incorporated into other useful imaging devices such as surgical goggles or intraoperative microscopes and/or viewers.

Preferably, the light source provides monochromatic (or substantially monochromatic) light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., Proc. Natl. Acad. Sci. USA 91:4887-4891, 1994; Ntziachristos et al., Proc. Natl. Acad. Sci. USA 97:2767-2772, 2000; and Alexander, J. Clin. Laser Med. Surg. 9:416-418, 1991. Information on lasers for imaging can be found, for example, at Imaging Diagnostic Systems, Inc., Plantation, FL and various other sources.

A high pass or bandpass filter can be used to separate optical emissions from excitation light. A suitable high pass or bandpass filter is commercially available from Omega Optical, Burlington, Vt.

In general, the light detection system can be viewed as including a light gathering/image forming component and a light detection/image recording component. Although the light detection system can be a single integrated device that incorporates both components, the light gathering/image forming component and light detection/image recording component will be discussed separately.

A particularly useful light gathering/image forming component is an endoscope. Endoscopic devices and techniques which have been used for in vivo optical imaging of numerous tissues and organs, including peritoneum (Gahlen et al., J. Photochem. Photobiol. B 52:131-135, 1999), ovarian cancer (Major et al., Gynecol. Oncol. 66:122-132, 1997), colon and rectum (Mycek etal., Gastrointest. Endosc. 48:390-394, 1998; and Stepp et al., Endoscopy 30:379-386, 1998), bile ducts (Izuishi et al., Hepatogastroenterology 46:804-807, 1999), stomach (Abe et al., Endoscopy 32:281-286, 2000), bladder (Kriegmair et al., Urol. Int. 63:27-31, 1999; and Riedl et al., J. Endourol. 13:755-759, 1999), lung (Hirsch et al., Clin Cancer Res 7:5-220, 2001), brain (Ward, J. Laser Appl. 10:224-228, 1998),esophagus, and head and neck regions can be employed in the practice of the present invention.

Other types of light gathering components useful in the invention are catheter-based devices, including fiber optics devices. Such devices are particularly suitable for intravascular imaging. See, e.g., Tearney et al., Science 276:2037-2039, 1997; and Circulation 94:3013, 1996.

Still other imaging technologies, including phased array technology (Boas et al., Proc. Natl. Acad. Sci. USA 91:4887-4891, 1994; Chance, Ann. NY Acad. Sci. 838:29-45, 1998), optical tomography (Cheng et al., Optics Express 3:118-123, 1998; and Siegel et al., Optics Express 4:287-298, 1999), intravital microscopy (Dellian et al., Br. J. Cancer 82:1513-1518, 2000; Monsky et al, Cancer Res. 59:4129-4135, 1999; and Fukumura et al., Cell 94:715-725, 1998), confocal imaging (Korlach et al., Proc. Natl. Acad. Sci. USA 96:8461-8466, 1999; Rajadhyaksha et al., J. Invest. Dermatol. 104:946-952, 1995; and Gonzalez et al., J. Med. 30:337-356, 1999) and fluorescence molecular tomography (Nziachristos et al., Nature Medicine 8:757-760, 2002; U.S. Pat. No. 6,615,063, PCT Application No. WO 03/102558, and PCT US/03/07579) can be employed in the practice of the present invention.

A suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a optical imaging system, and operating the system is within ordinary skill in the art.

### Diagnostic Methods

The methods of the invention can be used to determine a number of indicia, including tracking the localization of the fluorescent silicon nanoparticle imaging probe in the subject over time or assessing changes or alterations in the metabolism and/or excretion of the fluorescent silicon nanoparticle imaging probe in the subject over time. The methods can also be used to follow therapy for such diseases by imaging molecular events and biological pathways modulated by such therapy, including but not limited to determining efficacy, optimal timing, optimal dosing levels (including for individual patients or test subjects), and synergistic effects of combinations of therapy.

The invention can be used to help a physician or surgeon to identify and characterize areas of disease, such as arthritis, cancers and specifically colon polyps, or vulnerable plaque, to distinguish diseased and normal tissue, such as detecting tumor margins that are difficult to detect using an ordinary operating microscope, e.g., in brain surgery, help dictate a therapeutic or surgical intervention, e.g., by determining whether a lesion is cancerous and should be removed or non-cancerous and left alone, or in surgically staging a disease, e.g., intraoperative lymph node staging, sentinel lymph node mapping, or assessing intraoperative bleeding.

The methods of the invention can also be used in the detection, characterization and/or determination of the localization of a disease, especially early disease, the severity of a disease or a disease-associated condition, the staging of a disease, and monitoring and guiding various therapeutic interventions, such as surgical procedures, and monitoring drug therapy, including cell based therapies. The methods of the invention can also be used in prognosis of a disease or disease condition. Examples of such disease or disease conditions include inflammation (e.g., inflammation caused by arthritis, for example, rheumatoid arthritis), cancer (e.g., colorectal, ovarian, lung, breast, prostate, cervical, skin, brain, gastrointestinal, mouth, esophageal, bone), cardiovascular disease (e.g., atherosclerosis and inflammatory conditions of blood vessels, ischemia, stroke, thrombosis), dermatologic disease (e.g., Kaposi's Sarcoma, psoriasis), ophthalmic disease (e.g., macular degeneration, diabetic retinopathy), infectious disease (e.g., bacterial, viral, fungal and parasitic infections, including Acquired Immunodeficiency Syndrome), immunologic disease (e.g., an autoimmune disorder, lymphoma, multiple sclerosis, rheumatoid arthritis, diabetes mellitus), central nervous system disease (e.g., a neurodegenerative disease, such as Parkinson's disease or Alzheimer's disease), inherited diseases, metabolic diseases, environmental diseases (e.g., lead, mercury and radioactive poisoning, skin cancer), and bone-related disease (e.g., osteoporosis, primary and metastatic bone tumors, osteoarthritis). The methods of the invention can therefore be used, for example, to determine the presence of tumor cells and localization of tumor cells, the presence and localization of inflammation, including the presence of activated macrophages, for instance in atherosclerosis or arthritis, the presence and localization of vascular disease including areas at risk for acute occlusion (i.e., vulnerable plaques) in coronary and peripheral arteries, regions of expanding aneurysms, unstable plaque in carotid arteries, and ischemic areas. The methods and compositions of the invention can also be used in identification and evaluation of apoptosis, necrosis, hypoxia and angiogenesis.

### Dose

Ultimately, for in vivo human imaging a physician, radiologist or imaging technician or other technical personnel will decide the appropriate amount and dosage regimen based on the subject being imaged, the subject's age, weight, and disease state, and the location and type of tissue of interest in combination with imaging equipment parameters. Additionally, an effective amount can be that amount of fluorescent silicon nanoparticle imaging probe that is safe and efficacious in a human subject as determined and approved by a regulatory authority, such as the U.S. Food and Drug Administration.

The non-limiting examples provided herein, provide guidance in selecting the appropriate dose for non-human animal imaging and in vitro studies. The appropriate dose will be decided by the imaging technologist, radiologist or physician, using information such as tissue of interest, cells, tissues or animal subject being imaged, the subject's age, weight, and disease state, in combination with imaging equipment parameters.

Optical imaging modalities and measurement techniques include, but are not limited to, fluorescence imaging, luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; intravital imaging; two photon imaging; interferometry; coherence interferometry; diffluse optical tomography and fluorescence molecular tomography, and measurement of light scattering, absorption, polarisation, luminescence, fluorescence lifetime, quantum yield, and quenching.

The compositions and methods of the present invention can be used in combination with other imaging compositions and methods. For example, the methods of the present invention can be used in combination with other traditional imaging modalities such as X-ray, computed tomography (CT), positron emission tomography (PET), single photon computerized tomography (SPECT), and magnetic resonance imaging (MRI). For instance, the compositions and methods of the present invention can be used in combination with CT and MR imaging to obtain both anatomical and biological information simultaneously, for example, by co-registration of a tomographic image with an image generated by another imaging modality. In particular, the combination with MRI or CT is preferable, given the high spatial resolution of these imaging techniques. The compositions and methods of the present invention can also be used in combination with X-ray, CT, PET, SPECT and MR contrast agents or the fluorescent silicon nanoparticle imaging probes of the present invention may also contain components, such as iodine, gadolidium atoms and radioactive isotopes, which can be detected using CT, PET, SPECT, and MR imaging modalities in combination with optical imaging.

### Kits

The fluorescent silicon nanoparticle imaging probes described herein can be packaged as a kit, which may optionally include instructions for using the fluorescent silicon nanoparticle imaging probe in various exemplary applications. Non-limiting examples include kits that contain, e.g., a fluorescent silicon imaging probe in a powder or lyophilized form, and instructions for using the probe, including reconstituting the probe, dosage information, and storage information for in vivo and/or in vitro applications. Kits may optionally contain containers of fluorescent silicon nanoparticle imaging probes in a liquid form ready for use, or requiring further mixing with solutions for administration. For in vivo applications, the kit may contain the fluorescent silicon nanoparticle imaging probe in a dosage and form suitable for a particular application, e.g. a liquid in a vial, a topical creams, etc.

The kit can include optional components that aid in the administration of the unit dose to subjects, such as vials for reconstituting powder forms, syringes for injection, customized IV delivery systems, inhalers, etc. The kits may be supplied in either a container which is provided with a seal which is suitable for single or multiple puncturing with a hypodermic needle (e.g. a crimped-on septum seal closure) while maintaining sterile integrity. Such containers may contain single or multiple subject doses. Additionally, the unit dose kit can contain customized components that aid in the detection of the fluorescent silicon nanoparticle imaging probe in vivo or in vitro, e,g., specialized endoscopes, light filters. The kits may also contain instructions for preparation and administration of the compositions. The kit may be manufactured as a single use unit dose for one subject, multiple uses for a particular subject; or the kit may contain multiple doses suitable for administration to multiple subjects (“bulk packaging”). The kit components may be assembled in cartons, blister packs, bottles, tubes, and the like.

### EXAMPLES

The following non limiting examples demonstrate the synthesis of biocompatible silicon nanoparticles using various methods.

### Example 1a

Sodium metal (230 mg, Aldrich) was cut into small pieces under hexane and transferred to a 2-neck, oven dried 250 mL round bottom flask (RBF) flushed with nitrogen and containing of naphthalene (1.0 g, Aldrich) and a glass stir bar. The flask was evacuated and backfilled with nitrogen 3 times, then 20 mL of anhydrous THF (Aldrich) was added via syrnge. The mixture was stirred for 16 hours at room temperature (RT) resulting in a dark green solution of sodium naphthalenide. Silicon tetrachloride (224 uL, Aldrich) was dissolved in 30 mL anhydrous TBF in a nitrogen flushed, 2-neck, 500 mL RBF with a stirbar. The above sodium naphthalenide solution was then transferred to the flask rapidly via cannula at RT, resulting in the immediate formation of a cloudy brown suspension.

The brown suspension was reacted with 1.0 mL of water added rapidly by syringe. The cloudy brown suspension immediately turned a light, sandy brown color. The TUF was removed in vacuo and 40 mL of water was added. Naphthalene was removed by filtration through a 0.2μ membrane, resulting in an aqueous solution that exhibits bright blue fluorescence under irradiation at 366 nm. The nanoparticles were treated with HCl or buffer before use.

PLE/PL: λₘₐₓ excitation=336 nm; λₘₐₓ emission=460 nm

### Example 1b

Example 1a was repeated substituting 265 μL hexachlorodisilane for silicon tetrachloride. The product was reacted with 1.0 μL water as per Example 1 resulting in particles that exhibit bright blue fluorescence under irradiation at 366 nm. The nanoparticles were treated with HCl or buffer before use.

PLE/PL: λₘₐₓ excitation=336 nm; λₘₐₓ emission=460 nm.

### Example 2

Sodium metal (230 mg, Aldrich) was cut into small pieces under hexane and transferred to a 2-neck, oven dried 250 mL round bottom flask (RBF) flushed with nitrogen and containing of naphthalene (1.0 g, Aldrich) and a glass stir bar. The flask was evacuated and backfilled with nitrogen 3 times, then 20 mL of anhydrous THF (Aldrich) was added via syringe. The mixture was stirred for 16 hours at room temperature (RT) resulting in a dark green solution of sodium naphthalenide. Silicon tetrachloride (224 uL, Aldrich) was dissolved in 30 mL anhydrous THF in a nitrogen flushed, 2-neck, 500 mL RBF with a stirbar. The above sodium naphthalenide solution was then transferred to the flask rapidly via cannula at RT, resulting in the immediate formation of a cloudy brown suspension. Octanol (1.65 ml) was then added. Solvent was evaporated and the naphthalene was removed under vacuum with heating in a water bath at 50-60° C. The nanoparticles were treated with HCl or buffer before use. The resulting nanoparticles had the following optical properties:

PLE/PL: λₘₐₓ excitation=335 nml λₘₐₓ emission=430 nm.

### Example 3

Sodium metal (230 mg, Aldrich) was cut into small pieces under hexane and transferred to a 2-neck, oven dried 250 mL round bottom flask (RBF) flushed with nitrogen and containing of naphthalene (1.0 g, Aldrich) and a glass stir bar. The flask was evacuated and backfilled with nitrogen 3 times, then 20 mL of anhydrous THF (Aldrich) was added via syringe. The mixture was stirred for 16 hours at room temperature (RI) resulting in a dark green solution of sodium naphthalenide. Silicon tetrachloride (224 uL, Aldrich) was dissolved in 30 mL anhydrous THF in a nitrogen flushed, 2-neck, 500 mL RBF with a stirbar. The above sodium naphthalenide solution was then transferred to the flask rapidly via cannula at RT, resulting in the immediate formation of a cloudy brown suspension. Polyethylene glycol monomethyl ether (mPEG), 1 g (MW ˜350, Sigma) was then added and the solution was allowed to stir for 4 hours. A yellow suspension formed. The solution was filtered through a glass fritted filter to give a cloudy yellow filtrate. The nanoparticles were treated with HCl or buffer before use.

PLE/PL: λₘₐₓ excitation=403 nm; , emission=475 nm in alcohol.

Summary of Examples 1-3: These examples demonstrate that silicon nanoparticles can be produced from synthetic routes (rather than etched silicon wafers) and that coating agents can be used to “tune” the excitation wavelengths of the fluorescent silicon nanoparticles. In Example 3, the excitation wavelengths for the mPEG particles were longer than the water treated particles (up to 450 nm).

### Example 4

Magnesium silicide (115 mg, Aldrich) was placed in a 100 mL pressure vessel flushed thoroughly with nitrogen. 10 mL of hexane and 225 uL of bromine (Aldrich) were added and the vessel was sealed tightly. The vessel was placed in a sonication bath and sonicated for 2 hours, after which all of the bromine color had vanished. The sealed tube was cooled to 0° C. and carefully opened, releasing some pressure and a smoky vapor. A stir bar was placed in the flask and the suspension was stirred while 4 mL of methanol was slowly added under a strong stream of nitrogen. The resulting suspension was centrifuged in a 15 mL Falcon tube at 3,800 rpm for 15 minutes. The resulting orange, fluorescent supernatant containing the fluorescent silicon nanoparticles was decanted from the black solid at the bottom of the tube and filtered through a 0.45μ PTFE syringe filter (Acrodisc).

PLE/PL: λₘₐₓ excitation=445 nm; λₘₐₓ emission=515 nm.

### Example 5

The procedure of Example 4 was followed with the following modifications: After initial sonication, the flask was opened and the hexane was evaporated with a stream of nitrogen. 10 mL of dry ether was added to the flask. The flask was sealed and sonicated an additional 1 hour. 7.5 mL of methanol was added slowly under nitrogen and centrifuged as before.

Magnesium salts were removed from the filtered orange fluorescent methanol solution by passing 5-10 mL of the solution through a pasteur pipette plugged with a small piece of cotton and filled with 4 mL of anhydrous Na₂HPO₄.

PLE/PL: λₘₐₓ excitation=445 nm; λₘₐₓ emission=515 nm.

Summary of Examples 4 and 5: These Examples demonstrated that synthesis modifications change the optical properties of the resulting nanoparticles. The silicon nanoparticles in Examples 4 and 5 had excitation wavelengths longer than the nanoparticles produced in Examples 1-3 (445 nm vs. 335 nm for Examples 1-3); moreover, at 500 nm excitation, these particles retained 65% of their the emission intensity.

### Example 6

### Bromopropyl Silane Coated Nanoparticles

Silicon nanoparticles produced by the method of Li et al (Langmuir 19:8490-8496, 2003) with OH (oxidized) surface termination were used as the starting material. The nanoparticles supplied in ethanol were dried under vacuum with the aid of a heat gun (heat applied for 45 seconds), the flask being backfilled with dry nitrogen (yield, 9 mg dry).

100 μL of 3-bromopropyl trichlorosilane was added and the flask was sonicated for 15 seconds in a sonication bath to disperse the nanoparticles. 1.0 mL of dry toluene was added, and the solution was sonicated under nitrogen for 2-4 hours. The resulting nanoparticles were isolated by filtering through a 0.2μ teflon membrane filter. The resulting silane coated nanoparticles were washed with 2×2 mL of toluene, 2×2 mL of methanol and 2×2 mL of ether and dried on the filter. FTIR: 2936, 1433, 1299 cm⁻¹.

### Example 7

### Cyanopropyl Silane Coated-Nanoparticles

Silicon nanoparticles produced by the method of Li et al (Langmuir 19:8490-8496, 2003) with OH (oxidized) surface termination were used as the starting material. The nanoparticles supplied in ethanol were dried under vacuum with the aid of a heat gun (heat applied for 45 seconds), the flask being backfilled with dry nitrogen.

In this example, 100 μL of 3-trichlorosilyl butyronitrile was substituted for 3-bromopropyl trichlorosilane in Example 6 to produce another silane coated nanoparticle. FTIR: 2940, 2248, 1455, 1424, 1350 cm⁻¹.

### Example 8

### Aminopropyl Silane Coated Nanoparticles

Silicon nanoparticles produced by the method of Li et al (Langmuir 19:8490-8496, 2003) with H (reduced) surface termination were used as the starting material. The silicon nanoparticles (1 mL ethanolic dispersion) were suspended in 1 mL of neat 3-aminopropyl trimethoxysilane in a 50 mL RBF thoroughly flushed with nitrogen. The flask was sonicated for 1.5 hours, then kept at room temperature for 24 hours. Nanoparticles were isolated by filtration through a 0.2 mL teflon membrane filter. The nanoparticles were washed with 2×2 mL of toluene, 2×2 mL of methanol and 2×2 mL of ether and dried on the filter. The aminopropyl reagent resulted in complete quenching of photoluminescence.

### Example 9

### Mercaptopropyl Silane Coated Nanoparticles

Silicon nanoparticles produced by the method of Li et al (Langmuir 19:8490-8496, 2003) with OH (oxidized) surface termination were used as the starting material. The nanoparticles supplied in ethanol were dried under vacuum with the aid of a heat gun (heat applied for 45 seconds), the flask being backfilled with dry nitrogen.

The nanoparticles (4 mg) were suspended in 250 μL of neat 3-mercaptopropyl trimethoxysilane (Aldrich) in a 12 mL vial thoroughly flushed with nitrogen. The sealed vial was sonicated for 1.5 hours, then kept at room temperature for 24 hours. The resulting silane coated nanoparticles were isolated by filtration through a 0.2μ teflon membrane filter. The nanoparticles were washed with 2×2 mL of toluene, 2×2 mL of methanol and 2×2 mL of ether and dried on the filter.

FTIR: 2932, 1408, 1344 cm⁻¹

### Example 10

### Glucosamine Conjugated Silicon Nanoparticles

5 mg of iodoacetic acid, succinimidyl ester (Sigma) and 10 mg of glucosamine hydrochloride (Aldrich) were combined in 500 uL of 50% ethanol/10 mM phosphate buffer and the pH was adjusted to 8 by addition of about 25 uL of 1 M NaOH. The solution was kept at room temperature for 3 hours. 1 mg of mercaptopropyl silane coated nanoparticles from Example 9 were added. The solution was sonicated for 5 minutes to disperse the nanoparticles and left at room temperature for 15 hours. The glucosamine conjugated nanoparticles were separated on a 30 kDa MW cutoff filter membrane (Amicon). FTIR: 3268, 2930, 1651, 1537 cm⁻¹.

### Example 11

### Secondary Substitution: Mercaptoacetic Acid

Bromopropylsilane coated nanoparticles produced in Example 6 (2 mg) were placed in a flask under nitrogen with 500 uL mercaptoacetic acid (Aldrich) and 1.0 mL of methanol. The suspension was sonicated for 2 hours. Mercaptopropylsilane conjugated nanoparticles were isolated by filtration through a 0.2 μL teflon membrane filter. The nanoparticles were washed with 2×2 mL of methanol and 2×2 mL of ether and dried on the filter. FTIR: 3277, 2961, 1714, 1433, 1409 cm⁻¹ This example demonstrates that a second coating can be attached to coated nanoparticles.

### Example 12

### Peptide Conjugated Silicon Nanoparticles

Bromopropylsilane coated nanoparticles of Example 6 (0.5 mg) were placed in a flask under nitrogen with 300 μL of methanol and 2.1 mg of H-ArgGlyAspSerCys-OH [SEQ ID NO: 1] (Bachem). The suspension was sonicated for 2 hours and left at room temperature for 15 h. Nanoparticles were isolated by filtration through a 0.2 μl teflon membrane filter. The resulting peptide conjugated nanoparticles were washed with 2×2 mL of methanol and 2×2 mL of ether and dried on the filter. Alternatively, nanoparticles were isolated by ultrafiltration using a 30 kDa MW cut-off membrane (Millipore). Material was removed from the filter membrane with the aid of 2 seconds of sonication with a probe sonicator into 1× PBS with 10% (w/w) mannitol. Fluorescence of the aqueous suspension quenches with time, ˜3-5 h based on visual inspection. Dry nanoparticles retain fluorescence. FTIR: 3350, 2929, 1660, 1521, 1434 cm⁻¹.

### Example 13

### mPEG-Thiobutane Coated Silicon Nanoparticles

mPEG 4-mPEGthio-1-butene was synthesized as follows: 100 mg of mPEG thiol (MW=5000, Shearwater) was dissolved in 4 mL of 50/50 THF/methanol with 200 uL 4-bromo-1-butene and 100 μL of triethylamine. The mixture was kept under nitrogen at room temperature for 15 hours. The solvent and excess reagents were removed in vacuo.

Silicon nanoparticles produced by the method of Li et al (Langmuir 19:8490-8496, 2003) with H (reduced) surface termination (2 mg) were added to 10 mg of 4-mPEGthio-1-butene and 250 uL of IM ethylaluminum dichloride (Aldrich) and dispersed in 10 mL of 20% ethylene glycol dimethylether in diethyl ether under nitrogen. The suspension was sonicated for 15 minutes to disperse particles, then stirred at room temperature for 20 hours. 2 mL of methanol was added and the suspension was centrifuged at 3,500 rpm for 10 minutes. The supernatant was decanted off and the resulting solid was dispersed in 2 mL methanol, filtered on a 0.2 μL PTFE membrane and washed successively with 2 mL each of ether, methanol, ethanol, and ether again. The resulting mnPEG-thiobuatane coated silicon nanoparticles were then dried on the filter. FTIR: 2882, 1466, 1342, 1279 cm⁻¹.

### Example 14

### Peptide Conjugated Silicon Nanoparticles

Bromopropylsilane coated nanoparticles (2.0 mg) of Example 6 were placed in a 1.5 mL polystyrene vial under nitrogen with 250 uL of methanol and 10 mg of Ac-ArgArgArgArgGlyArgArgArgArgGlyCys-NH₂ (SEQ ID NO: 2) Tufts University Core Facility). The suspension was sonicated for 2 hours and left at RT for 15 h. Nanoparticles were isolated by filtration through a 0.2 μl teflon membrane filter. The resulting peptide conjugated nanoparticles were washed with 2×1 mL of methanol and 2×1 mL of ether and dried on the filter. FTIR: 3342, 2888, 1656, 1435, 1349 cm⁻¹.

### Example 15

### Peptide Conjugated Silicon Nanoparticles

Bromopropylsilane coated nanoparticles (2.0 mg) from Example 6 were placed in a 1.5 mL polystyrene vial under nitrogen with 500 uL of methanol and 10 mg of Ac-ArgGlyAspSerCysArgGlyAspSer-NH₂ (SEQ ID NO: 3) (Tufts University Core Facility). The suspension was sonicated for 2 hours and left at room temperature for 15 h. Nanoparticles were isolated by filtration through a 0.2 μl teflon membrane filter. The resulting peptide coated nanoparticles were washed with 2×1 mL of methanol and 2×1 mL of ether and dried on the filter. FTIR: 3279, 2940, 1657, 1543, 1410 cm⁻¹.

### Example 16

### EGF-Conjugated Silicon Nanoparticles

Human EGF (Sigma, 0.2 mg) and iodoacetic acid, succinimidyl ester (Sigma, 2.5 mg) were combined in 200 uL 0.1 M sodium bicarbonate with 5% ethanol. The solution was sonicated for 15 seconds and vortexed for 60 seconds and kept at room temperature for 15 hours. The solution was filtered through a 0.45 μl teflon syringe filter to remove undissolved material, diluted to 1 mL with water and concentrated to about 50 μL over a 5 kDa MW cutoff filter membrane (Amicon) at 3,000 rpm for 30 minutes. An additional 1 mL of water was added and the solution was concentrated again in the same manner. The material was diluted to 200 uL with 1× PBS and 0.5 mg of the mercaptopropyl silane coated nanoparticles of Example 9 was added. The solution was sonicated for one hour, then allowed to react for 15 hours at room temperature. The resulting EGF conjugated nanoparticles were separated from unreacted protein using a 30 kDa MW cut-off filter membrane (Amicon) (0.5 mL capacity), washed with 0.5 mL of distilled water and dried under vacuum. FTIR: 3317, 2932, 1653, 1536, 1406 cm⁻¹.

### Example 17

### Hexane Cbated Silicon Nanoparticles

Silicon nanoparticles produced by the method of Li et al (Langmuir 19:8490-8496, 2003) with H (reduced) surface termination (2 mg) were dispersed in 1.0 mL anhydrous diethyl ether. 500 μL of 1-hexene (Aldrich) and 50 μL of 1.0 M ethylaluminum dichloride in hexanes was added. The solution was kept under a nitrogen atmosphere and sonicated 10 minutes to disperse the nanoparticles and then stirred at RT for 15 hours. 0.5 mL of methanol were then added and the resulting hexane coated nanoparticles were filtered on a 0.2μ PTFE membrane, washed with 2 mL each of methanol, water, ethanol and diethyl ether and dried on the membrane. FTIR: 2924, 1460 cm⁻¹.

### Example 18

### mPEG Coated Silicon Nanoparticles

Bromopropylsilane coated nanoparticles (2.0 mg), from Example 6 were placed in a 1.5 mL polystyrene vial under nitrogen with 250 μL of methanol and 10 mg of mPEG-SH, MW 5 kDa (Shearwater). The suspension was sonicated for 2 hours and left at room temperature for 15 h. Nanoparticles were isolated by filtration through a 0.2μ teflon membrane filter. The resulting mPEG coated nanoparticles were washed with 2×1 mL of methanol and 2×1 mL of ether and dried on the filter. FTIR: 2879, 1466, 1342 cm⁻¹.

### Example 19

### Fluorescence-Stabilized Nanoparticles

4-(mPEGthio)butane nanoparticles of Example 13 were formulated in 10% (w/v) mannitol in aqueous PBS. The nanoparticles retained>90% of their fluorescence after 7 days versus uncoated nanoparticles which generally lose their fluorescence after several hours in aqueous media.

### Example 20

### In vivo Imaging studies

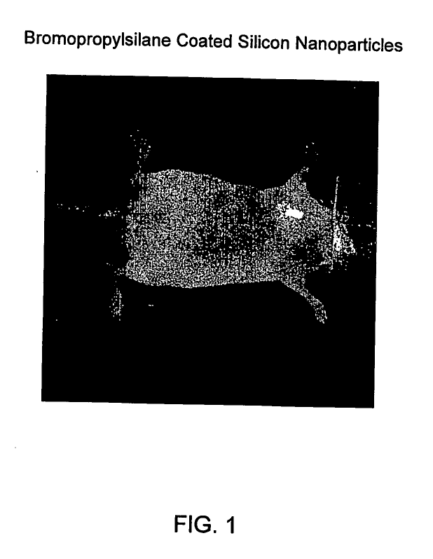

Six-week old female NU/NU nuBR nude (Charles River Laboratories) mice received a subcutaneous injection (between the first and second left mammary glands) of a fluorescent silicon nanoparticle imaging probe (100 μl) using a 27 gauge (1 cc) syringe. The fluorescent silicon nanoparticle imaging probe was prepared by suspending bromopropyl silane-coated silicon nanoparticles of Example 6 at a concentration of 3 mg/ml in PBS containing 20% (w/v) mannitol (Aldrich). For in vivo detection, mice were anesthetized by inhalation of halothane mixed in oxygen. Mice were then placed in a small animal imaging system (Kodak Scientific Imaging). This system includes a 150 W halogen light source to provide broad-spectrum white light and a removable 465 nm excitation filter for IS2000MM (CAT#8197709, Kodak) mounted between the halogen bulb and a fiber optic bundle, to create a uniform excitation source in the 465 nm range. Two mirrors direct the light path to the imaging object and/or to the detector. Photons emitted by the fluorescent object being imaged are selected using a 700 nm long pass filter which removes scattered excitation photons, partially due to the wide wavelength separation of the filter set. The bandpass excitation filter is mounted on a removable holder and the emission filter on a flywheel, to allow for easy switching between fluorescent imaging and white light imaging, without moving the animal. The fluorescence signal is detected by a low light level CCD camera and the signal output recorded on a PC computer as a 12 bit data image using Kodak ID imaging software. Acquisition time was 1 minute (4×15 sec added). The resulting image is shown in FIG. 1.

### Example 21

### In vivo Imaging Studies

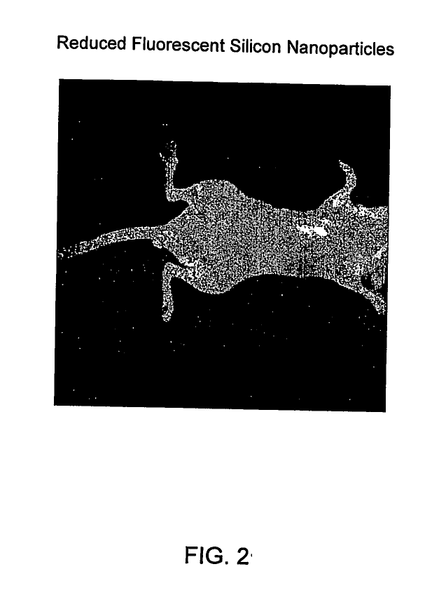

Six-week old female NU/NU nuBR nude (Charles River Laboratories) mice received a subcutaneous injection (between the first and second left mammary glands) of a fluorescent silicon nanoparticle imaging probe (100 μl) using a 27 gauge (1 cc) syringe. The fluorescent silicon nanoparticle imaging probe was prepared by suspending reduced silicon nanoparticles produced by the method of Li et al (Langmuir 19:8490-8496, 2003) at a concentration of 7 mg/ml in 10% (v/w) mannitol (Aldrich) and 3% DMSO in PBS. For in vivo detection, mice were anesthetized by inhalation of halothane mixed in oxygen. Mice were then placed in a small animal imaging system (Kodak Scientific Imaging). This system includes a 150 W halogen light source to provide broad-spectrum white light and a removable 465 nm excitation filter for IS2000MM (CAT#8197709, Kodak) mounted between the halogen bulb and a fiber optic bundle, to create a uniform excitation source in the 465 nm range. Two mirrors direct the light path to the imaging object and/or to the detector. Photons emitted by the fluorescent object being imaged are selected using a 700 nm long pass filter which removes scattered excitation photons, partially due to the wide wavelength separation of the filter set. The bandpass excitation filter is mounted on a removable holder and the emission filter on a flywheel, to allow for easy switching between fluorescent imaging and white light imaging, without moving the animal. The fluorescence signal is detected by a low light level CCD camera and the signal output recorded on a PC computer as a 12 bit data image using Kodak ID imaging software. Acquisition time was 1 minute (4×15 sec added). The resulting image is shown in FIG. 2.

### Example 22

### In vivo Imaging Studies

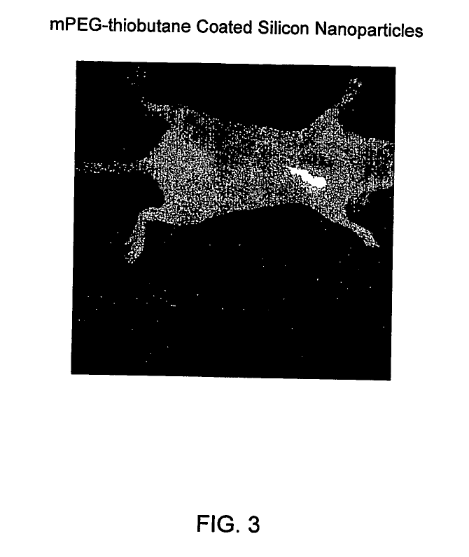

Six-week old female NU/NU nuBR nude (Charles River Laboratories) mice received a subcutaneous injection (between the first and second left mammary glands) of a fluorescent silicon nanoparticle imaging probe (100 μl) using a 27 gauge (1 cc) syringe. The fluorescent silicon nanoparticle imaging probe was prepared by suspending mPEGthiobutane coated silicon nanoparticles of Example 13 at a concentration of 5 mg/ml in PBS containing 10% (v/w) mannitol (Aldrich). For in vivo detection, mice were anesthetized by inhalation of halothane mixed in oxygen. Mice were then placed in a small animal imaging system (Kodak Scientific Imaging). This system includes a 150 W halogen light source to provide broad-spectrum white light and a removable 465 nm excitation filter for IS2000MM (CAT#8197709, Kodak) mounted between the halogen bulb and a fiber optic bundle, to create a uniform excitation source in the 465 nm range. Two mirrors direct the light path to the imaging object and/or to the detector. Photons emitted by the fluorescent object being imaged are selected using a 700 nm long pass filter which removes scattered excitation photons, partially due to the wide wavelength separation of the filter set. The bandpass excitation filter is mounted on a removable holder and the emission filter on a flywheel, to allow for easy switching between fluorescent imaging and white light imaging, without moving the animal. The fluorescence signal is detected by a low light level CCD camera and the signal output recorded on a PC computer as a 12 bit data image using Kodak ID imaging software. Acquisition time was 1 minute (4×15 sec added). The resulting image is shown in FIG. 3.

### Example 23

### In vivo Imaging Studies

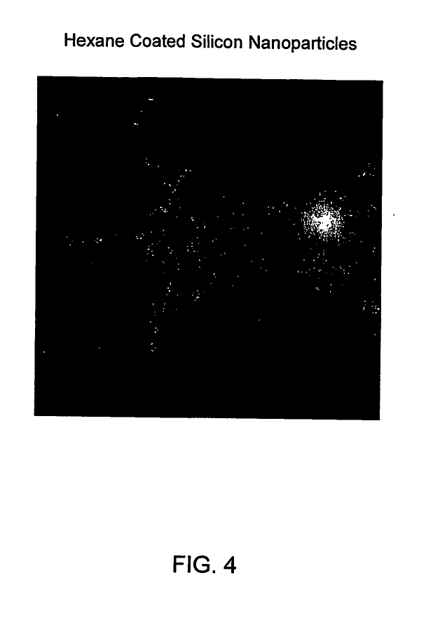

Six-week old female NU/NU nuBR nude (Charles River Laboratories) mice received a subcutaneous injection (between the first and second left mammary glands) of a fluorescent silicon nanoparticle imaging probe (100 μl) using a 27 gauge (1 cc) syringe. The fluorescent silicon nanoparticle imaging probe was prepared by suspending hexane coated silicon nanoparticles of Example 17 at a concentration of 5 mg/ml in PBS containing 10% (v/w) mannitol (Aldrich). For in vivo detection, mice were anesthetized by inhalation of halothane mixed in oxygen. Mice were then placed in a small animal imaging system (Kodak Scientific Imaging). This system includes a 150 W halogen light source to provide broad-spectrum white light and a removable 465 nm excitation filter for IS2000MM (CAT#8197709, Kodak) mounted between the halogen bulb and a fiber optic bundle, to create a uniform excitation source in the 465 nm range. Two mirrors direct the light path to the imaging object and/or to the detector. Photons emitted by the fluorescent object being imaged are selected using a 700 nm long pass filter which removes scattered excitation photons, partially due to the wide wavelength separation of the filter set. The bandpass excitation filter is mounted on a removable holder and the emission filter on a flywheel, to allow for easy switching between fluorescent imaging and white light imaging, without moving the animal. The fluorescence signal is detected by a low light level CCD camera and the signal output recorded on a PC computer as a 12 bit data image using Kodak ID imaging software. Acquisition time was 1 minute (4×15 sec added). The resulting image is shown in FIG. 4.

While this invention has been particularly shown and described with references to preferred embodiments thereof, it will be understood by those skilled in the art that various changes in form and details may be made therein without departing from the scope of the invention encompassed by the appended claims.

What is claimed is:

1. A biocompatible fluorescent silicon nanoparticle comprising a fluorescent silicon nanoparticle and a biocompatible coating.

2. The biocompatible nanoparticle of claim 1, flilrer comprising a biomolecule chemically linked to the biocompatible fluorescent silicon nanoparticle.

3. The biocompatible nanoparticle of claim 1, wherein the fluorescent silicon nanoparticle has an absorption and emission maxima between about 300 nm and about 1,200 nm.

4. The biocompatible nanoparticle of claim 1, wherein the size of the fluorescent silicon nanoparticle is about 0.5 nm to about 10 mn in diameter.

5. The biocompatible fluorescent silicon nanoparticle of claim 1, wherein the biocompatible coating is a polymer.

6. The biocompatible fluorescent silicon nanoparticle of claim 1, wherein the biocompatible coating is a silane.

7. The biocompatible fluorescent silicon nanoparticle of claim 5, wherein the polymer is selected from the group consisting of carboxymethyl dextran, dextrans, polyethylene glycol, and biocompatible graft copolymers.

8. The biocompatible fluorescent silicon nanoparticle of claim 5, wherein the polymer is selected from the group consisting of polyamino acids, polyethyleneamines, polysaccharides, polyamidoamines, polyacrylic acids, polyalcohols, polyoxyethylene sorbitan esters, polyoxyethytene and polyoxypropylene derivatives, polyoxyl stearates, polycaprolactones, polyanhydrides, polyalkylcyanoacrylates, polyglycerol surfactants, polycaprolactones, polyanhydrides, polymethylmethacrylate polymers, starch derivatives, dextran and derivatives thereof, fatty acids and derivatives thereof, polyethylene glycol, methoxypolyethylene glycol, methoxypolypropylene glycol, polyethylene glycol-diacid, and polyethylene glycol monoamine.

9. The biocompatible fluorescent silicon nanoparticle of claim 2, wherein the biomolecule is selected from the group consisting of proteins, peptides, antibodies or antigen binding fragments thereof, cell receptor ligands, polysaccharides, cell receptors, enzyme substrates, enzyme cofactors, biotin, hormones, neurohormones, neurotransmitters, growth factors, cytokines, lymphokines, lectins, toxins, carbohydrates, membrane or transmembrane translocation signal sequences, and nuclear translocation signal sequences.

10. The biocompatible fluorescent silicon nanoparticle of claim 1, wherein the biocompatible fluorescent silicon nanoparticle is a fluorescent silicon nanoparticle imaging probe.

11. The biocompatible fluorescent silicon nanoparticle of claim 10, wherein the fluorescent silicon nanoparticle imaging probe is activated after target interaction.

12. The biocompatible fluorescent silicon nanoparticle of claim 10, wherein the fluorescent silicon nanoparticle imaging probe has a high binding affinity to a target.

13. A method of in vivo optical imaging, the method comprising:
(a) administering to a subject fluorescent silicon nanoparticle imaging probes of claim 10;
(b) allowing time for the fluorescent silicon nanoparticle imaging probes to contact a biological target;
(c) illuminating the target with light of a wavelength absorbable by the fluorescent silicon nanoparticle imaging probes; and
(d) detecting the optical signal emitted by the fluorescent silicon nanoparticle imaging probes.

14. The method of claim 13, wherein steps (a)-(d) are repeated at predetermined intervals thereby allowing for evaluation of emitted signal of the fluorescent silicon nanoparticle imaging probes in the subject over time.

15. The method of claim 13, wherein the signal emitted by the fluorescent silicon nanoparticle imaging probes is used to construct an image.

16. The method of claim 15, wherein the image is co-registered with an image obtained by magnetic resonance or computed tomography imaging.

17. The method of claim 13, wherein the subject is an animal.

18. The method of claim 13, wherein the subject is a human.

19. The method of claim 13, wherein the illuminating and detecting steps are done using an endoscope, catheter, tomographic system, hand-held optical imaging system, surgical goggles, or intraoperative microscope.

20. The method of claim 13, wherein the presence, absence, or level of optical signal emitted by the fluorescent silicon nanoparticle imaging probes is indicative of a disease state.

21. The method of claim 13, wherein the method is used in the early detection or staging of a disease.

22. The method of claim 13, wherein the method is used in monitoring or determining a therapeutic course of action for a treatment of a disease.

23. The method of claim 22, wherein the therapeutic course of action is surgical.

24. The method of claim 22, wherein the therapeutic course of action comprises administration of a drug therapy.

25. The method of claim 13, wherein the method is used to assess the effect of one or more drug therapies on a disease state.

26. The method of claim 20, wherein the disease is selected from the group consisting of cancer, cardiovascular diseases, neurodegenerative diseases, immunologic diseases, autoimmune diseases, metabolic diseases, inherited diseases, infectious diseases, bone diseases, and environmental diseases.

27. The method of claim 13, wherein in step (a), more than one distinguishable fluorescent silicon nanoparticle imaging probe is administered to the subject and wherein in step (d) more than one optical signal emitted by the fluorescent silicon nanoparticle imaging probe target is detected.

28. An in vitro optical imaging method, the method comprising:
(a) contacting a sample with the probes of claim 10;
(b) allowing time for the probes to become activated or bind to the biological target of interest in the sample;
(c) optionally, removing the unbound probes;
(d) illuminating the target with light of a wavelength absorbable by the fluorescent silicon nanoparticle imaging probes; and
(e) detecting the optical signal emitted by the fluorescent silicon nanoparticle imaging probes.

29. The method of claim 28, wherein the sample is selected from the group consisting of primary cells, cell cultures, tissue, and cytospin samples.

30. The method of claim 28, wherein in step (a), more than one distinguishable imaging probe is administered to the sample and wherein in step (d) more than one target is detected simultaneously in a sample.


SEQUENCE LISTING (3 sequences)

SEQ ID NO: 1 - protein, 5 aa, Artificial Sequence
  Synthetic Polypeptide
RGDSC

SEQ ID NO: 2 - protein, 12 aa, Artificial Sequence
  Synthetic Polypeptide
RRRARGRRRRGC

SEQ ID NO: 3 - protein, 10 aa, Artificial Sequence
  Synthetic Polypeptide
RGASSCRGDS